(19) United States
(12) Patent Application Publication
(10) Pub. No.: US 2004/0170592 A1
(43) Pub. Date: Sep. 2, 2004

(54) Use of multiple emulsions as skin protection products

(76) Inventors: Marcel Veeger, Goch (DE); Andreas Klotz, Grevenbroich (DE); Edeltraut Blaeser, Krefeld (DE); Silke Seidl, Willich (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,525

(22) PCT Filed: Apr. 30, 2002
(86) PCT No.: PCT/EP02/04751

(30) Foreign Application Priority Data
Apr. 30, 2001 (DE) 101-20-927.4

(51) Int. Cl. A61K 7/075; A61K 7/08

(52) U.S. Cl. 424/70.31

(57) ABSTRACT

Use of multiple W/OW-emulsions in the production of silicon-free skin protection agents, especially useful against harmful aqueous substances, characterized in that said W/OW emulsions are produced by using an emulsifier mixture consisting of polyolpoly-12-hydroxystearates combined with an alkyl and/or alkylene glucoside and a fatty alcohol and/or partial glycerides. The invention also relates to corresponding skin care products.

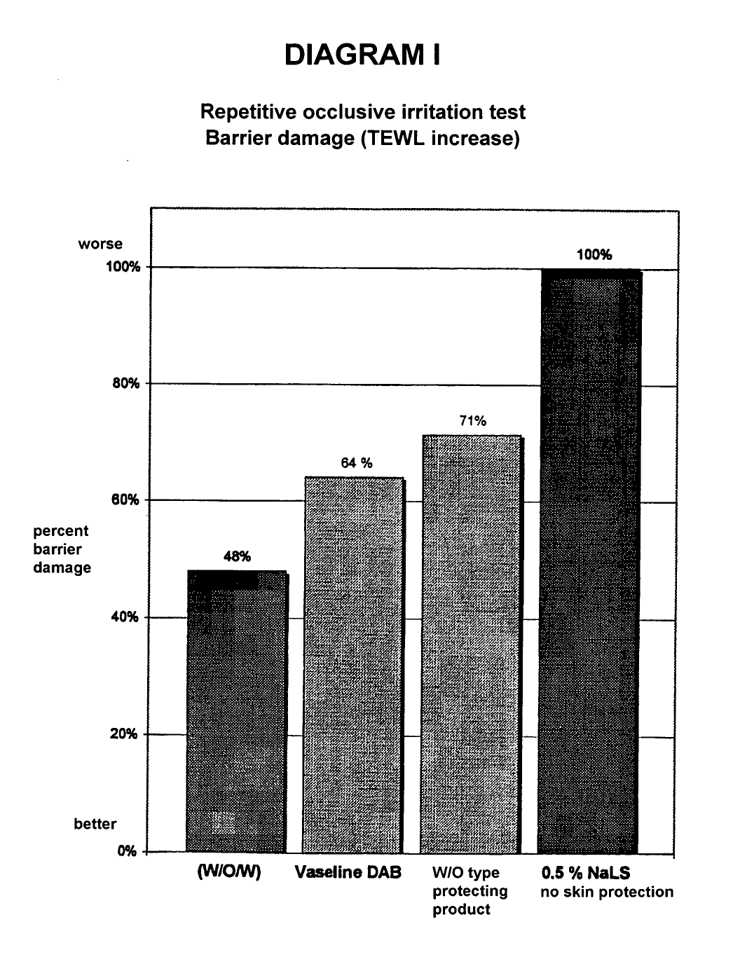

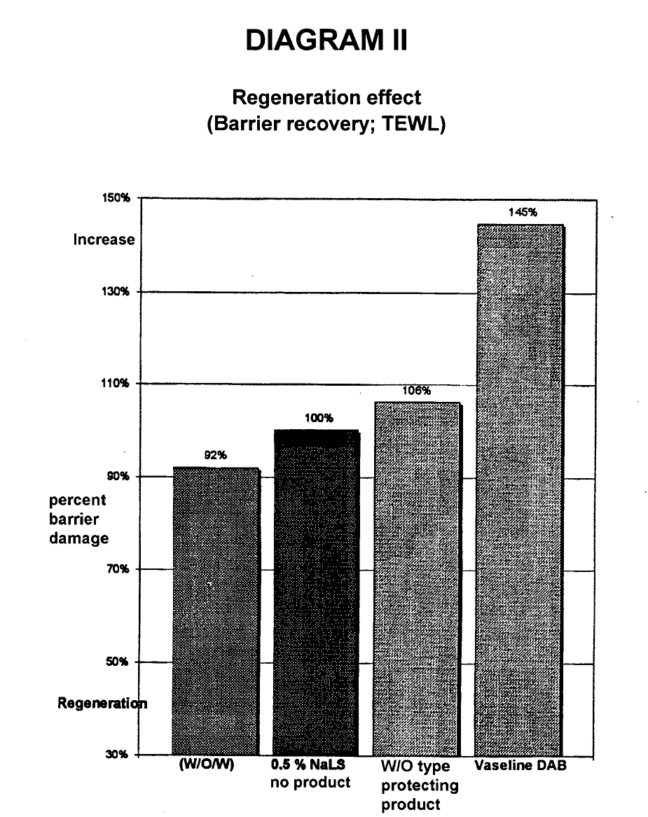

The invention relates to the use of multiple emulsions as skin protecting products to protect against aqueous noxae, and to corresponding products.

Skin protecting products are protective agents against lesions of the skin which can be caused e.g. by effects of the weather, by water and aqueous solutions, chemicals and particularly industrial contaminations such as hazardous or heavily contaminating working substances. Such skin protecting products which, in particular, are intended to prevent skin exposure to working substances, cover the skin with a so-called barrier film acting as a protective barrier against irritating and harmful substances.

Above all, common barrier agents are paraffin hydrocarbons such as mineral oils, vaseline, etc., but also, mineral and vegetable waxes, including silicone oils and silicone waxes. Skin protecting preparations are commercially available in the form of most various types of formulations, the most important being skin ointments, skin creams, skin lotions, skin oils, and skin gels. Skin creams and skin lotions are predominantly based on O/W (oil-in-water) or W/O (water-in-oil) type emulsions. Thus, the major components of the oil phase (as well as fat or lipid phase) can be fatty alcohols, fatty acids, fatty acid esters, waxes, vaseline, paraffins, as well as other fats and oils of mainly natural origin.

Among other things, the aqueous phase may include water-soluble care agents having a moisture-regulating or moisture-retaining effect.

The question which of the above-mentioned types of emulsions should be used in a skin protecting product will primarily depend on the protective purpose to be pursued by means of such a product, or on the type of working substance against which the product should offer protection.

Apart from the O/W or W/O type emulsions conventionally used in skin protecting products, multiple emulsions used in the production of cosmetic and pharmaceutical products have also been described.

Such multiple emulsions are emulsions of emulsions, the most important representatives of which, i.e., multiple water/oil/water (W/O/W) and oil/water/oil (O/W/O) emulsions, have been extensively described in the patent literature.

Thus, DE 41 31 678 A1 describes multiple emulsions which can be used in cosmetic skin care products, but also, in medical topical formulations.

Water and aqueous solutions of moderately skin-irritating substances may give rise to cumulative toxic contact dermatitis (wearing dermatosis) upon repeated skin contact over a prolonged period of time. To protect the skin against such strain, it is common practice to use water-insoluble barrier preparations forming a protective film on the skin. However, such preparations involve the disadvantage of impairing the natural release of water vapor via the skin. As a result of the associated accumulation of heat and moisture, the acceptance of using such products will be reduced in employees exposed to daily and frequent contact with aqueous substances noxious to the skin.

More acceptance have hydrophilic O/W (oil-in-water) type formulations which achieve their protecting effect through high amounts of included silicone compounds.

Silicones such as silicone oils and silicone waxes are excellent barrier agents having particularly high stability in skin protecting agents. Thus, they are thermally stable and have extraordinary resistance when exposed to corrosive chemicals. Moreover, such silicone preparations are strongly hydrophobic and harmless to the skin, because they are physiologically tolerable, i.e., non-deleterious, as well as kind to the skin. The low surface tension of silicone oils allows easy distribution thereof on the skin. Another advantage is that silicone layers on the skin—in contrast to paraffins, vaseline etc. —do not involve any risk of accumulation of heat on the skin.

Thus, DE 41 31 678 A1 also describes a skin protecting emulsion which contains cyclomethicone as silicone compound, the W/O/W emulsion being obtained by using ethoxylated fatty alcohols as emulsifiers.

However, such preparations containing silicones are disadvantageous in that these preparations may leave residues on objects, e.g. on materials or workpieces, when bringing such workpieces to further operation by hand. Thus, in the event of workpieces to be painted, for example, employees cannot use such silicone-containing skin protecting agents because these silicone residues, which are difficult to remove, represent a massive interference during the further processing of these workpieces, such as painting or vulcanizing. Thus, especially in the automotive, lacquer and rubber-processing industries, silicone-containing skin protecting products cannot be employed despite their outstanding protective effect and acceptance.

Consequently, there is a great demand for skin protecting agents free of silicone oil, which would exhibit a comparably good effectiveness in protection against water and aqueous, skin-irritating solutions.

The object of the present invention was therefore to develop such skin protecting agents free of silicone which, in addition, would have high cosmetic acceptance like e.g. skin care products.

Surprisingly, it has been found that it is possible to obtain such skin protecting agents produced using W/O/W emulsions formed by means of a mixture of emulsifiers comprised of polyol poly-12-hydroxystearates in combination with an alkyl- and/or alkyleneglucoside and a fatty alcohol and/or partial glycerides and optionally other co-emulsifiers, particularly at least one ethoxylated dipolyhydroxystearate as co-emulsifier.

The production of such multiple emulsions or W/O/W emulsions developed under the aspect of skin care has been described in DE 196 12 084 A1 which hereby is fully incorporated by reference.

According to the invention, for a skin protecting agent, the mixture of emulsifiers is employed in amounts of from 1 to 25 wt.-%, preferably from 5 to 15 wt.-%, relative to the overall composition of the skin protecting agent.

Here, the mixture of emulsifiers includes at least one polyol poly-12-hydroxy-stearate as emulsifier component, preferably in an amount of from 1 to 5 wt.-%, relative to the overall composition of the skin protecting agent, with polyglycerol poly-12-hydroxystearates commercially available under the brand of Dehymuls® PGPH from Henkel KGaA, Dusseldorf, being particularly preferred as emulsifier component.

Advantageously, further co-emulsifiers can be added to the mixture of emulsifiers. Preferably, at least one ethoxylated dipolyhydroxystearate, particularly at a dosage of from 0.1 to 1.0 wt.-%, also relative to the overall composition of the skin protecting agent, can be added to the mixture of emulsifiers, with PEG-30 dipolyhydroxystearates being particularly preferred as co-emulsifiers, which are commercially available under the designation of ARLACEL P135. Other co-emulsifiers which can be used are hydrophilic emulsifiers well-known to those skilled in the art, which can likewise be added to the mixture of emulsifiers at a dosage of from 0.1 to 1.0 wt.-%, relative to the total amount of agent. In this context, PEG-40 stearate may be mentioned as example.

The development of the multiple W/O/W emulsion has shown that high cosmetic acceptance (comparable to a mild care cream) in addition to high effectiveness against aqueous noxae can be present if the amount of non-polar oils does not exceed 20 wt.-%, preferably 15 wt.-%, and more preferably 10 wt.-%.

Oils normally usable in cosmetic and pharmaceutical formulations, which cover the skin with a protective or barrier film, are employed as non-polar oils, particularly the above-mentioned paraffin hydrocarbons, as well as mineral oils, e.g. vaseline etc., including mineral and vegetable waxes, provided they do not give rise to undesirable residues on materials and workpieces, as is the case with preparations containing silicones, for example.

Owing to the polarity of these oils, an excellent protective effect against hydrophilic substances noxious to the skin is achieved. As supplements, these non-polar oils may include additives such as isopropyl palmitate or isopropyl myristate or other additives enhancing the spreadability or strength, which additives are well-known to those skilled in the art.

Moreover, it was found that addition of 0.1 to 5 wt.-%, preferably 0.5 to 2 wt.-%, and more preferably 0.5 to 1.5 wt.-% of bisabolol can result in an improvement of the barrier, especially in the event of pre-damaged skin, thereby achieving effective support of skin regeneration. Similarly, addition of natural vegetable tanning agents, preferably in an amount of from 0.1 to 5 wt.-% relative to the total amount of skin protecting agent, is also advantageous, with Hamamelis Virginia as tanning agent being particularly preferred.

Owing to the inventive combination of emulsion base, non-polar oils and particularly bisabolol, it was possible to combine the beneficial effects of skin protection, skin regeneration and cosmetic acceptance in a single formulation. This was surprising because said non-polar oils—which normally tend to involve an impeding effect as to skin regeneration and cosmetic acceptance, but represent a valuable component in skin protection—in such multiple W/O/W emulsions result in beneficial effects regarding skin protection, skin regeneration and cosmetic acceptance.

In particular, the skin protecting agents which, according to the invention, are produced using multiple emulsions provide excellent protection against aqueous noxae, thus ensuring good skin protection even when working under wet conditions according to TRGS 531 (Technical Regulations for Hazardous Substances, Germany). Consequently, such skin protecting agents can be employed with advantage in work-places involving aqueous strain of the skin, e.g. in the food-processing, metal-processing, rubber-processing industries, in hospitals, in agriculture and forestry, but also, in leisure, hobby and domestic activities, e.g. garden work and dish washing.

In addition, there is no interference with work processes by residues of silicone compounds e.g. on materials or workpieces.

The following examples are intended to illustrate the present invention. The figures in the examples represent percentages by weight, relative to the total weight of the respective preparations.

Examples of Preferred Skin Protecting Formulations (All Figures in % by Weight)

| | A | B | C |
|---|---|---|---|
| Mixture of C16/18 alkylglycosides and fatty alcohol | 3.00 | 4.00 | 5.00 |
| Polyglycerol poly-12-hydroxystearate | 2.00 | 3.00 | 3.50 |
| PEG-30 dipolyhydroxystearate | 0.50 | 0.50 | 1.00 |
| PEG-40 stearate | 0.50 | | |
| Paraffin oil | 6.00 | 8.00 | 10.00 |
| Vaseline white DAB (German Pharmacopoeia) | 1.00 | 2.00 | 3.00 |
| Isopropyl palmitate | 3.00 | 3.00 | 3.00 |
| C12/15 Alkyl benzoates | 1.50 | 2.00 | 3.00 |
| Cetylstearyl alcohol | 1.00 | 1.00 | 1.00 |
| Stearic acid | 1.50 | 3.00 | 4.00 |
| Bisabolol | 0.50 | 1.00 | 2.00 |
| 1,2-Propanediol | 1.00 | 2.00 | 3.00 |
| Preservative | q.s. | q.s. | q.s. |
| Vegetable tanning agent | 0.50 | 1.00 | 2.00 |

| | |
|---|---|
| Water | ad 100 |

Test Methods:

1) Skin Protecting Effect

The skin protecting effect was studied in the following “in vivo” test model in order to test the protective effect of skin protecting products against the model noxious substance sodium laurylsulfate (0.5% in water, referred to as NaLS hereinafter) as reference substance for aqueous substances noxious to the skin.

The skin protecting preparations are coated on the skin twice a day over 1 week, and the model noxious substances are subsequently applied thereon. The skin reaction is assessed visually each day, and important parameters are determined using measuring methods having no effect on the skin (e.g. TEWL). Up to 7 test products can be tested in each test series.

The test group consists of at least 5 males and females with healthy skin and aged from 18 to 60.

Days 1 to 4:

The skin condition of test areas marked on the volar forearm is determined (instrumentally via TEWL). The TEWL0 is determined prior to treatment. 0.1 g of skin protecting product is applied on the volar side of the forearm, observing a randomization on an area of 3 cm×3 cm (9 cm²), and rubbed into the skin with a rubber fingerstall or latex glove. An area including model irritant with no skin protecting product serves as negative control.

10 minutes after applying the product, any excess is dabbed away with a cellulose cloth. Thereafter, Finn chambers including noxious substance are applied on all test areas for 30 minutes (12 mm chamber: 50 μl; 18 mm chamber: 200 μl). Subsequently, the Finn chambers are removed, and the arm is rinsed with tap water. Three hours ±0.5 hours after the first application, the application of skin protection and noxious substance is repeated.

On day 5, final assessment of the skin reaction is effected, with no further application of product (TEWL5).

Evaluation is performed using the following formula:

(mean TEWL5_{skin protection}−mean TEWL0_{skin protection})/

(mean TEWL5_{skin protection}−mean TEWL0_{skin protection})×100%

The control value (with 0.5% NaLS, no skin protection///mean TEWL5_{no skin protection}−mean TEWL0_{no skin protection}) is set equal to 100% barrier damage. This furnishes the following result for various skin protecting products, which is illustrated graphically in diagram |:

The percent barrier damage, measured using the TEWL in a repetitive occlusive irritation test (according to Contact Dermatitis 2000, 42, 336-343), can be reduced to 48% by additional use of the W/O/W formulations described above. When using a commercially available skin protecting formulation, a reduction in the dermal changes to 71% merely can be achieved. When using vaseline DAB, which has a very poor acceptance, the dermal changes can be reduced to 64%.

Accordingly, the W/O/W formulations of the invention allow for the production of skin protecting products having a significant effect against aqueous noxae. This was surprising in that commercially available O/W formulations, the outer phase of which consists of water, do not exhibit any effect against aqueous noxae.

2) Skin Care Effect

Moreover, the inventive skin protecting agents free of silicone, which are produced using the multiple W/O/W emulsions described herein, also have regenerative care effects as demonstrated by means of the test method described below:

Similarly, the method that is used is an “in vivo” test model to test the protective effect of skin protecting products. The required skin lesion is induced by means of sodium laurylsulfate (0.5% in water).

The skin protecting preparations are coated twice a day over 1 week on the previously irritated skin. The skin reaction is assessed visually each day, and important parameters are determined using measuring methods having no effect on the skin (e.g. TEWL). Up to 7 test products can be tested in each test series. The test group consists of at least 5 males and females with healthy skin and aged from 18 to 60.

Days 1 to 4:

The skin condition of test areas marked on the volar forearm is determined (instrumentally via TEWL). The TEWL0 is determined prior to treatment.

Thereafter, Finn chambers including noxious substance are applied on all test areas for 30 minutes (12 mm chamber: 50 μl; 18 mm chamber: 200 μl). Following removal of the Finn chambers, the arms are rinsed with tap water and dabbed dry.

Subsequently, 0.1 g of skin protecting product is applied on the volar side of the forearm, observing a randomization on an area of 3 cm×3 cm (9 cm²), and rubbed into the skin with a rubber fingerstall or latex glove. An area including model irritant with no skin protecting product serves as negative control.

10 minutes after applying the product, any excess is dabbed away with a cellulose cloth. Three hours ±0.5 hours after the first application, the application of skin protection and noxious substance is repeated.

On day 5, final assessment of the skin reaction is effected, with no further application of product (TEWL5).

Evaluation

As above, the following formula has been developed for the barrier damage:

(mean TEWL5_{skin protection}−mean TEWL0_{skin protection})/ (mean TEWL5_{no skin protection}−mean TEWL0no skin protection)×100%

The control value, 0.5% NaLS with no skin protection (mean TEWL5_{no skin protection}−mean TEWL0_{no skin protection}), is set equal to 100% barrier damage. According to the regeneration test model, the percent regeneration effect R is expressed as follows:

R=100% −percent barrier damage, i.e.,

R=100% −(mean TEWL5_{skin protection}−mean TEWL0_{skin protection})/ (mean TEWL5_{no skin protection}−mean TEWL0_{no skin protection})×100%

This furnishes the following result for various skin protecting products, which also is illustrated graphically in diagram II:

The percent barrier damage, measured using the TEWL in a regeneration model, can be reduced to 92% when using the W/O/W formulations described above. This corresponds to a regeneration effect of 8%. When using a commercially available W/O skin protecting formulation or vaseline, the barrier damage even increased in this model, being 106.2% and 145%, respectively, resulting in a negative regeneration effect or a barrier damage increase by 6.2% and 45%, respectively.

As demonstrated by the results determined, the inventive skin protecting formulations including the W/O/W formulations described above have a significant regenerative (care) effect.

Accordingly, the W/O/W formulations are remarkable for their skin protecting effect against the NaLS aqueous model noxa employed, and for their regenerative effect in cases of barrier damage.

According to INCI, the commercially available W/O protecting formulation used against aqueous noxae in the above tests, which represents the present state of the art, is composed as follows:

Water, Paraffinum liquidum, Petrolatum, talc, zinc oxide, glycerin, methylglucose dioleate, caprylic/capric triglyceride, isohexadecane, PEG-45/dodecyl glycol co-polymer, C18-36 acid triglyceride, magnesium stearate, C12-15 alkyl benzoate, magnesium sulfate, perfume, sodium bischlorophenylsulfamine, phenoxyethanol, methyl paraben, ethyl paraben, propyl paraben, butyl paraben, isobutyl paraben, lactic acid.

1. Use of multiple W/O/W emulsions in the production of skin protecting agents free of silicone, particularly against aqueous noxae, characterized in that the W/O/W emulsions are formed using a mixture of emulsifiers comprised of polyol poly-12-hydroxystearates in combination with an alkyl- and/or alkyleneglucoside and a fatty alcohol and/or partial glycerides.

2. The use according to claim 1, characterized in that the mixture of emulsifiers is employed in amounts of from 1 to 25 wt.-%, relative to the overall composition of the skin protecting agent.

3. The use according to claims 1 to 2, characterized in that the mixture of emulsifiers is employed in amounts of from 5 to 15 wt.-%, relative to the overall composition of the skin protecting agent.

4. The use according to claims 1 to 3, characterized in that the mixture of emulsifiers includes at least one polyol poly-12-hydroxystearate as emulsifier component.

5. The use according to claims 1 to 4, characterized in that the mixture of emulsifiers includes polyglycerol poly-12-hydroxystearates as polyol poly-12-hydroxystearate emulsifier component.

6. The use according to claims 1 to 5, characterized in that the mixture of emulsifiers additionally includes at least one ethoxylated dipolyhydroxystearate.

7. The use according to claims 1 to 6, characterized in that PEG-30 dipolyhydroxystearates are employed as ethoxylated dipolyhydroxystearates.

8. The use according to claims 1 to 7, characterized in that the amount of non-polar oils in the skin protecting agent does not exceed 20 wt.-%, preferably 15 wt.-%, and more preferably 10 wt.-%.

9. The use according to claims 1 to 8, characterized in that bisabolol is included in the skin protecting agent in an amount of from 0.1 to 5 wt.-%, preferably from 0.5 to 2 wt.-%, and more preferably from 0.5 to 1.5 wt.-%.

10. The use according to claims 1 to 9, characterized in that natural vegetable tanning agents are included in the skin protecting agent in an amount of from 0.1 to 2 wt.-%, preferably 0.3 to 1 wt.-%, and more preferably from 0.4 to 0.8 wt.-%.

11. Skin protecting agents free of silicone, particularly against aqueous noxae, which agents can be obtained from W/O/W emulsions formed using a mixture of emulsifiers comprised of polyol poly-12-hydroxystearates in combination with an alkyl- and/or alkyleneglucoside and a fatty alcohol and/or partial glycerides.

12. The skin protecting agents according to claim 11, characterized in that the mixture of emulsifiers is employed in amounts of from 1 to 25 wt.-%, relative to the overall composition of the skin protecting agent.

13. The skin protecting agents according to claims 11 to 12, characterized in that the mixture of emulsifiers is employed in amounts of from 5 to 15 wt.-%, relative to the overall composition of the skin protecting agent.

14. The skin protecting agents according to claims 11 to 13, characterized in that the mixture of emulsifiers includes at least one polyol poly-12-hydroxystearate as emulsifier component.

15. The skin protecting agents according to claims 11 to 14, characterized in that the mixture of emulsifiers includes polyglycerol poly-12-hydroxystearates as polyol poly-12-hydroxystearate emulsifier component.

16. The skin protecting agents according to claims 11 to 15, characterized in that the mixture of emulsifiers additionally includes at least one ethoxylated dipolyhydroxystearate.

17. The skin protecting agents according to claims 11 to 16, characterized in that PEG-30 dipolyhydroxystearates are employed as ethoxylated dipolyhydroxystearates.

18. The skin protecting agents according to claims 11 to 17, characterized in that the amount of non-polar oils in the skin protecting agent does not exceed 20 wt.-%, preferably 15 wt.-%, and more preferably 10 wt.-%.

19. The skin protecting agents according to claims 11 to 18, characterized in that bisabolol is included in the skin protecting agent in an amount of from 0.1 to 5 wt.-%, preferably from 0.5 to 2 wt.-%, and more preferably from 0.5 to 1.5 wt.-%.

20. The skin protecting agents according to claims 11 to 19, characterized in that natural vegetable tanning agents are included in the skin protecting agent in an amount of from 0.1 to 2 wt.-%, preferably 0.3 to 1 wt.-%, and more preferably from 0.4 to 0.8 wt.-%.
